(19) United States
(12) Plant Patent Application Publication
(10) Pub. No.: US 2016/0242341 P1
(43) Pub. Date: Aug. 18, 2016

(54) Grapevine named 'SORELI'

(50) ;

(71) Applicants: Università degli Studi di Udine, Udine UD (IT); Istituto di Genomica Applicata, Udine UD (IT)

(72) Inventors: Simone Diego CASTELLARIN, Vancouver (CA); Guido CIPRIANI, Faedis (IT); Gabriele DI GASPERO, Cividale del Friuli UD (IT); Michele MORGANTE, Tricesimo UD (IT); Enrico PETERLUNGER, Codroipo UD (IT); Raffaele TESTOLIN, Udine (IT)

(73) Assignees: Università degli Studi di Udine, Udine UD (IT); Istituto di Genomica Applicata, Udine UD (IT)

(21) Appl. No.: 14/545,112

(22) Filed: Mar. 27, 2015

(30) Foreign Application Priority Data
Feb. 13, 2015 (QZ) 2015/0361

(51) Int. Cl. A01H 5/00

(52) U.S. Cl. PLT/207

(57) ABSTRACT

A new and distinct variety of grapevine, primarily suited to the growing conditions of the temperate regions, and characterized by its medium-strong vigor; horizontal growth habit; medium-sized, wedge-shaped to circular-shaped leaves; very low to low weight berry clusters; berries with slightly firm flesh, neutral taste, and no flesh coloration; primarily used for wine; and resistance to winter temperatures (to −24° C.), resistance to downy mildew, and tolerance to powdery mildew.

### LATIN NAME OF THE GENUS AND SPECIES OF THE PLANT CLAIMED

Vitis×vinifera (hybrid)

### VARIETY NAME

‘SORÉLI’

### BACKGROUND OF THE INVENTION

The present invention relates to a new and distinct summer/fall bearing grapevine variety, botanically known as Vitis×vinifera (hybrid), and hereinafter referred to by the name ‘SORÉLI’.

The new grapevine ‘SORÉLI’ is a product of a controlled breeding program conducted by the inventors in Udine, Italy. The objective of the breeding program was to develop a new grapevine variety particularly characterized by resistance to cold (<−20° C.), resistance to downy mildew (Plasmopara viticola), and tolerance to powdery mildew (Uncinula necator).

The new grapevine ‘SORÉLI’ originated from a cross made by the inventors in 2002 in Udine, Italy. The female or seed parent is the grapevine variety, Vitis vinifera cv. ‘Tocai Friulano’ (unpatented), and the male or pollen parent is the grapevine variety, Vitis cv. ‘20/3’ (Bianca×SK77-4/5)(unpatented).

The new grapevine ‘SORÉLI’ was discovered and selected by the inventor as a single flowering plant within the progeny of the stated cross in a controlled environment in 2002 in Udine, Italy. Asexual reproduction of the new grapevine variety by grafting was first performed in February 2004 in Rauscedo, Friuli Venezia Giulia region, Italy, and has demonstrated that the combination of characteristics as herein disclosed for the new cultivar are firmly fixed and retained through successive generations of asexual reproduction. The new cultivar reproduces true to type.

### SUMMARY OF THE INVENTION

‘SORÉLI’ is primarily adapted to the climate and growing conditions of the temperate regions with average yearly temperature about 13° C., minimum winter temperature about −21° C., annual rainfall around 700-1500 mm of rain (e.g. North-Eastern Italy, Friuli). This region provides the necessary year-round temperatures required for it to produce and maintain a strong vigorous plant with consistent fruit production from April through November on primocanes and in the ensuing year from April through November on the floricanes.

The following traits have been repeatedly observed and are determined to be unique characteristics of ‘SORÉLI’, which in combination distinguish this grapevine plant as a new and distinct variety.
1. Medium-strong vigor;
2. Horizontal growth habit;
3. Medium wedge-shaped to circular shaped leaves with dark green upper surfaces and, green lower surfaces and medium texture;
4. Very low to low weight, medium-dense, conical-shaped berry clusters with two middle size wings; green-yellow berries with slightly firm flesh, neutral taste, and no flesh coloration; primarily used for wine; and early maturity (late August in Middle Friuli, North-Eastern Italy);
5. Primocanes and floricanes with elliptic cross section, brownish color, no lenticels, and medium-sized internodes (diameter: 8 mm);
6. Early harvesting time (late August in Middle Friuli, North-Eastern Italy); and
7. Resistance to winter temperature (−24° C.), resistance to downy mildew, and tolerance to powdery mildew.

Plants of the new grapevine ‘SORÉLI’ differ from plants of the parents, Vitis vinifera ‘Tocai Friulano’ (unpatented) and Vitis cross ‘20/3’ (unpatented), in the following characteristics described in Table 1.

TABLE 1
Comparison with parent varieties

| | | Female Parent | Male Parent |
|---|---|---|---|
| Charac- | New Cultivar | ‘Tocai Friulano’ | ‘20/3’ |
| teristic | ‘SORÉLI’ | (unpatented) | (unpatented) |
| vigor: | medium-strong | strong | medium |
| growth | horizontal | semi-erect | semi-erect |
| habit | | | |
| leaf | medium, dark | medium size, light | Medium size, light |
| | green color (up- | green color (upper | green color (upper |
| | per surface), | surface), green | surface), green color |
| | green color (low- | color (lower sur- | (lower surface), no |
| | er surface), no | face), very few | hairs in both |
| | hairs in both sur- | hairs in both sur- | surfaces, wedge- |
| | faces, wedge- | faces, circular | shaped-kidney- |
| | shaped to circular | shape, medium | shaped, medium |
| | shape, medium | texture | texture |
| | texture | | |
| cluster | Very low-low | low weight, con- | low weight, conical, |
| | weight, conical | ical, with 1-2 | with 1-2 wings, |
| | shape with 1-2 | wings, dense, ber- | medium dense, ber- |
| | middle size wings, | ry skin with green- | ry skin with green- |
| | medium dense, | yellow color, soft | yellow color, |
| | berry skin with | flesh, neutral taste, | slightly firm flesh, |
| | green-yellow col- | no flesh coloration | neutral taste, |
| | or, slightly firm | | no flesh coloration |
| | flesh, neutral taste, | | |
| | no flesh coloration | | |
| primo- | elliptic cross sec- | circular cross sec- | oblate cross section, |
| cane and | tion, brownish col- | tion, brownish col- | brownish color, |
| floricane | or, lenticels: ab- | or, lenticels: ab- | lenticels: absent, |
| | sent, medium in- | sent, short-medium | medium internodes, |
| | ternodes, diameter: | internodes, diam- | diameter small |
| | small (about 8 mm) | eter small (about | (about 8 mm) |
| | | 8 mm) | |
| harvest- | early (last decade | Early-medium | Early (last decade |
| ing time | of August in Mid- | (early September, | August, Middle |
| | dle Friuli, North- | Middle Friuli, | Friuli, North- |
| | Eastern Italy), | North-Eastern | Eastern Italy) |
| | | Italy) | |
| resis- | resistant to winter | Average resistance | resistant to winter |
| tances | temperature | to winter tempera- | temperature not |
| | (−24° C.), resistant | ture (−15° C.), sus- | checked, resistant |
| | to downy mildew, | ceptible to downy | to downy mildew, |
| | tolerant to powdery | mildew, susceptible | resistant to pow- |
| | mildew. | to powdery mildew | dery mildew |

Of the many commercial cultivars known to the present inventor, the most similar to the new grapevine ‘SORÉLI’ is the female parent ‘Tocai Friulano’, to which a comparison has been provided above.

### BRIEF DESCRIPTION OF THE PHOTOGRAPHS

The accompanying photographs illustrate the overall appearance of the new grapevine ‘SORÉLI’ showing the colors as true as is reasonably possible with colored reproductions of this type. Colors in the photographs may differ slightly from the color values cited in the detailed botanical description, which accurately describe the color of ‘SORÉLI’.

FIG. 1A and FIG. 1B show a typical fruit cluster of ‘SORÉLI’ taken on Aug. 30, 2012, in Udine, Italy.

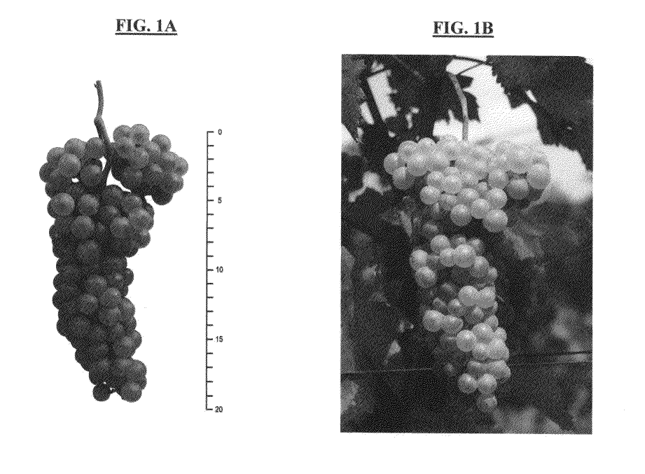

FIG. 2 shows a typical mature leaf (upper surface, left and lower surface, right) of ‘SORÉLI’ taken on May 31, 2012, in Udine, Italy.

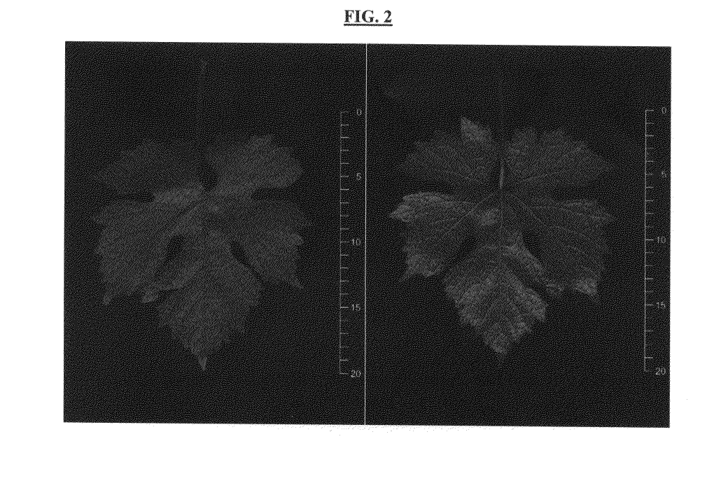

FIG. 3 shows a typical mature vine of ‘SORÉLI’ taken on Aug. 6, 2012.

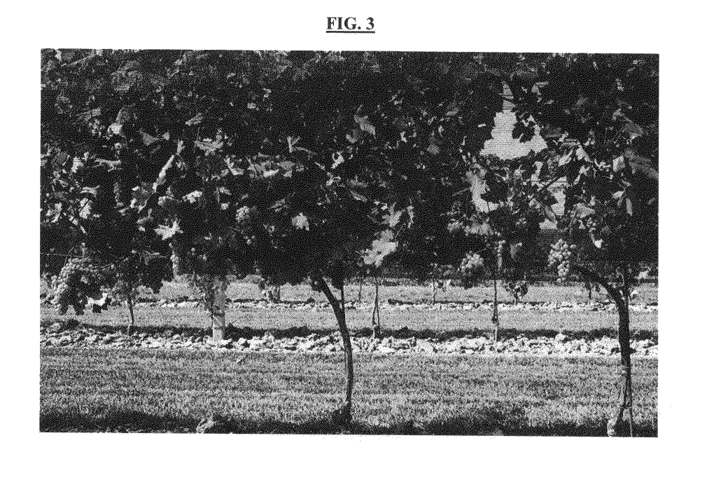

FIG. 4 shows a close-up view of typical mature fruit of ‘SORÉLI taken on Aug. 30, 2012, in Udine, Italy

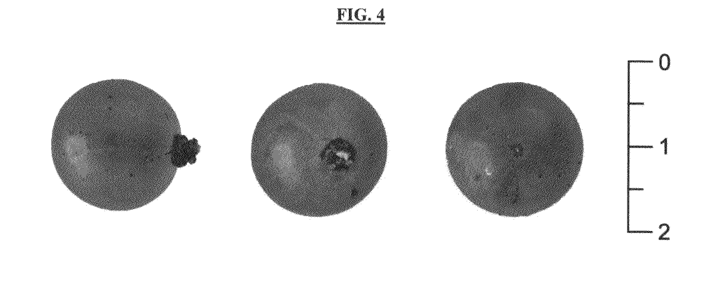

### DETAILED BOTANICAL DESCRIPTION

The following description of ‘SORÉLI’ unless otherwise noted, is based on observations taken during the 2011, 2012, and 2013 growing seasons(s) in Udine, Italy and Fossalon di Grado (GO), Italy, from plants dug from a nursery located in, Rauscedo (PN), Italy during the beginning of December 2007 and planted approximately 16 to 20 weeks later in Udine, Italy and Fossalon di Grado (GO), Italy. The phenotypical descriptions and color designations stated for the new variety may vary, depending upon variations in environmental factors, including weather (temperature, humidity and light intensity), day length, soil type, location and cultural conditions. ‘SORÉLI’ has not been observed under all possible environmental conditions.

| | | |
|---|---|---|
| | | ‘TOCAI |
| | | FRIULANO’ |
| Characteristic | ‘SORÉLI’ | (unpatented) |

GENERAL

| | | |
|---|---|---|
| Resistance to | Resistant to downy | Susceptible to downy |
| pest/disease | mildew, tolerant to | mildew and to powdery |
| | powdery mildew | mildew |
| Resistance to | Resistant to winter | Resistant to winter |
| natural elements | cold down to −24° C. | cold down to −15° C. |

VINE

| | | |
|---|---|---|
| Vigor | Medium-strong | strong |
| Trunk diameter | 6 cm (6 yrs-old plant) | 6.5 |
| Bark | | |
| color | Dark brown | Dark brown |
| underbark color | Light brown | Light brown |
| texture | n.a. | n.a. |
| Canes | | |
| length | Internode length | Internode length |
| | cm 12 | about cm 10 |
| width | 8 mm | 8 mm |
| Shoots | | |
| shape | Erected, cross section: | Semi-erected, cross sec- |
| | elliptic, surface: with | tion: circular, surface: |
| | stripes, nodes and inter- | smooth, nodes and inter- |
| | nodes without hairs | nodes without hairs |
| color | internodes with red and | Internodes with green |
| | green pigmentation on | color on ventral and dorsal |
| | both sides, nodes with | side, nodes with green ven- |
| | red and green pigmen- | tral and dorsal side |
| | tation on both sides | |
| Tendrils | | |
| form | bifid or trifid | bifid |
| color | Green | Green |
| texture | Normal | normal |
| number | 2 or <2 consecutive | 2 or <2 consecutive |
| Buds | | |
| size | average | average |
| shape | round | round |
| color | Light brown | Light brown |
| number | 2/node | 2/node |
| time of budbreak | early | early-medium |

LEAVES

| | | |
|---|---|---|
| Size | medium | medium |
| Number of leaflets | 5 | 5 |
| Glossiness | Medium | medium |
| Cross section shape | revolute | V-shaped |
| Color (immature) | | |
| Upper surface | green | Light green |
| Under surface | green | green |
| Color (mature) | | |
| Upper surface | green | Medium green |
| Under surface | green | Darker green |
| Petiole | | |
| Length (cm) | 5.5 | 6.0 |
| Color (upper | green | green |
| surface) | | |
| Color (under | green | green |
| surface) | | |
| Stipule orientation | n.a. | n.a. |

FLOWERS

| | | |
|---|---|---|
| Flowering period | End of May | End of May |
| (time of beginning | | |
| of flowering) | | |
| Sex | hermaphrodite | hermaphrodite |
| Size | Average | average |
| Diameter (cm) | 0.3 (stamens) | 0.3 (stamens) |
| Fragrance | average | average |
| Flower number (at | n.a. | n.a. |
| 3^{rd} node from tip of | | |
| lateral mean and | | |
| range) | | |
| Petals | | |
| Length (cm) | n.a. | n.a. |
| Width (cm) | n.a. | n.a. |
| Overall shape | calyptra | calyptra |
| Color (immature) | green | green |
| Upper surface | | |
| Under surface | | |
| Color (mature) | green | green |
| Upper surface | | |
| Under surface | | |
| Sepals | | |
| Length (cm) | n.a. | n.a. |
| Width (cm) | n.a. | n.a. |
| Overall shape | n.a. | n.a. |
| Color (immature) | n.a. | n.a. |
| Upper surface | | |
| Under surface | | |
| Color (mature) | n.a. | n.a. |
| Upper surface | | |
| Under surface | | |
| Pedicel | | |
| Length (mm) | 7 | 7 |
| Color | green | green |

FRUIT

| | | |
|---|---|---|
| Primocane time of | August 30th | September 7th |
| fruiting (1^{st} pick) | | |
| Clusters | | |
| cluster weight | g 170 | g 230 |
| cluster shape | cylindrical | conical |
| cluster length | long (mm 190) | medium |
| avg. berries per | 160 | 220 |
| cluster | | |
| avg. clusters per | 2 | 2 |
| shoot | | |
| Berries | | |
| Berry size | Very low to low (g 1.5) | Very low to low (g 1.8) |
| Berry length (cm) | 1.3 | 1.50 |
| Berry width (cm) | 1.3 | 1.50 |
| Overall shape of | globose | globose |
| berry | | |
| Berry Texture | slightly firm | soft |
| Berry Color | green | green |
| (immature) | | |
| Berry Color | green | green |
| (mature) | | |
| Soluble solids (%) | 22.7 | 21.1 |
| Titratable acidity | 5.0 | 5.7 |
| (as g/L tartaric | | |
| acid) | | |
| Sugar/acid ratio | 4.5 | 3.7 |
| Firmness | Slightly firm | soft |
| Seeds | 2-3 | 2-3 |
| Skin cracking? | no | rarely |
| Juice color | transparent | transparent |
| Berry taste | Neutral | neutral |
| Eating quality | n.a. | n.a. |
| Berry uses | wine | wine |
| Shipping quality | n.a. | n.a. |

What is claimed is:

1. A new and distinct grapevine, referred to as ‘SORÉLI, as herein described and illustrated by the characteristics set forth above.
